(19) United States
(12) Patent Application Publication
(10) Pub. No.: US 2024/0058624 A1
(43) Pub. Date: Feb. 22, 2024

(54) MEDICAL DEVICES, RADIATION ASSEMBLIES THEREOF, AND CONTROL METHODS THEREOF

(71) Applicant: SHANGHAI UNITED IMAGING HEALTHCARE CO., LTD., Shanghai (CN)

(72) Inventors: Jian ZHANG, Shanghai (CN); Xiaobin LI, Shanghai (CN); Cheng NI, Shanghai (CN)

(73) Assignee: SHANGHAI UNITED IMAGING HEALTHCARE CO., LTD., Shanghai (CN)

(21) Appl. No.: 18/451,068

(22) Filed: Aug. 16, 2023

(30) Foreign Application Priority Data
Aug. 16, 2022 (CN) 202210980010.0

(51) Int. Cl. A61N 5/10

(52) U.S. Cl. CPC A61N 5/1082; A61N 2005/1061

(57) ABSTRACT

The present disclosure provide a device. The device may include: a pedestal; a rotation base disposed on the pedestal; an imaging component disposed on the rotation base; and a treatment component disposed on the rotation base. The imaging component and the treatment component may be disposed on a same side of the rotation base, and the imaging component and the treatment component may be configured to rotate around a first rotation axis of the rotation base.

### CROSS-REFERENCE TO RELATED APPLICATIONS

This application claims priority of Chinese Application No. 202210980010.0, filed on Aug. 16, 2022, the contents of which are hereby incorporated by reference.

### TECHNICAL FIELD

The present disclosure relates to the field of medical devices, and in particular, to medical devices, radiation assemblies thereof, and control methods thereof.

### BACKGROUND

At present, in the treatment of tumors and other diseases, the integration of a computed tomography (CT) device and a radiation therapy (RT) device has attracted widespread attention. During a treatment process, the CT device is configured to monitor a target area in real-time, and adaptively adjust radiotherapy conditions according to the monitored organ position and volume change, such that a ray transmitter of the RT device can accurately locate the target area, so as to significantly improve the curative effect of radiotherapy. However, the current CT device and RT device are limited by rotation angles, leading that rays of the RT device can only be irradiated in one plane, resulting in incomplete treatment for patients or causing damage to the patient's target area and surrounding tissues, and increasing the probability of recurrence and other sequelae. Accordingly, it is necessary to adjust the patient's position to match the radiation range of the RT device, resulting in the treatment process with a long time and low efficiency.

Therefore, it is desirable to provide an efficient medical device that includes the CT device and the RT device and control methods thereof.

### SUMMARY

An aspect of the present disclosure provides a device. The device may include a pedestal; a rotation base disposed on the pedestal; an imaging component disposed on the rotation base; and a treatment component disposed on the rotation base. In some embodiments, the imaging component and the treatment component may be disposed on a same side of the rotation base, and the imaging component and the treatment component may be configured to rotate around a first rotation axis of the rotation base.

In some embodiments, an isocenter of the imaging component and an isocenter of the treatment component may be at a same position.

In some embodiments, the imaging component and the treatment component may be located at a same rotation unit of the rotation base.

In some embodiments, the device may further include a first drive component. The first drive component may be configured to drive the rotation base to rotate around the first rotation axis of the rotation base.

In some embodiments, the device may further include a rotation component disposed between the pedestal and the rotation base. In some embodiments, the imaging component and the treatment component may be configured to rotate around a second rotation axis parallel with the pedestal and the rotation base by the rotation component.

In some embodiments, an angle between the second rotation axis and a surface of the pedestal may range from 0 to 5°.

In some embodiments, a rotation angle of the rotation base rotating, by the rotation component, around the second rotation axis may range from 5° to 45°.

In some embodiments, a rotation angle of the rotation base rotating, by the rotation component, around the second rotation axis may range from 5° to 60°.

In some embodiments, the rotation component may include a first support unit and a second support unit disposed on the pedestal, and a first rocker arm and a second rocker arm disposed on the rotation base. In some embodiments, the first rocker arm may be rotatably connected with the first support unit, and the second rocker arm may be rotatably connected with the second support unit.

In some embodiments, the device may further include a second drive component. The second drive component may be configured to drive the rotation base to rotate around the second rotation axis.

In some embodiments, the second drive component may include a pneumatic pusher. In some embodiments, an end of the pneumatic pusher may be connected with the pedestal, and another end of the pneumatic pusher may be connected with the rotation base.

In some embodiments, the pedestal may be configured to rotate around a third rotation axis. In some embodiments, the third rotation axis may be perpendicular to a surface of the pedestal.

In some embodiments, the imaging component and the treatment component may be configured to rotate around the third rotation axis along with the rotation of the pedestal

In some embodiments, a rotation angle of the pedestal around the third rotation axis may be less than or equal to 90°.

In some embodiments, a rotation angle of the pedestal around the third rotation axis may be less than or equal to 120°.

In some embodiments, the device may further include a third drive component. The third drive component may be configured to drive the pedestal to rotate around the third rotation axis.

In some embodiments, the pedestal may include a stator and a rotor, the third drive component may include a motor and a conveyor. In some embodiments, the motor may be connected with the conveyor, and the conveyor may be wounded on the rotor.

In some embodiments, the rotation base may include a first rotation unit and a second rotation unit. In some embodiments, the treatment component may be disposed on the first rotation unit, the imaging component may be disposed on the second rotation unit, and the second rotation unit may be rotatable with respect to the first rotation unit.

In some embodiments, the imaging component may include includes an imaging radiation source, and the treatment component may include a treatment radiation source. In some embodiments, the imaging radiation source and the treatment radiation source may be disposed on the rotation base at intervals.

Another aspect of the present disclosure provides a device for a medical device. The device may include a pedestal; a rotation base disposed on the pedestal; an imaging component disposed on the rotation base; a treatment component disposed on the rotation base; and a rotation component disposed between the pedestal and the rotation base. In some embodiments, the imaging component and the treatment component may be configured to rotate around a rotation axis parallel with the pedestal and the rotation base by the rotation component.

Another aspect of the present disclosure provides a device for a medical device. The device may include a pedestal; a rotation base disposed on the pedestal; an imaging component disposed on the rotation base; and a treatment component disposed on the rotation base. In some embodiments, the imaging component and the treatment component may be configured to rotate around a rotation axis. In some embodiments, the rotation axis may be perpendicular to a surface of the pedestal.

Another aspect of the present disclosure provides a method. The method may be implemented on a computing device including at least one processor and a storage device. In some embodiments, the method may include determining one or more target positions of at least one of an imaging component or a treatment component of a device. In some embodiments, the device may include a rotation base and a pedestal. The method may further include controlling the at least one of the imaging component or the treatment component to rotate around at least one of a first rotation axis, a second rotation axis, and a third rotation axis based on the one or more target positions; and controlling the imaging component to perform imaging or the treatment component to generate radiotherapy rays. In some embodiments, the first rotation axis may be of the rotation base, the second rotation axis is parallel with the pedestal and the rotation base, and the third rotation axis is perpendicular to a surface of the pedestal.

Another aspect of the present disclosure provides a system. The system may include one or more processors, and a memory configured to store one or more programs. When the one or more programs are executed by the one or more processors, the one or more processors are made to implement operations. The operation may include determining one or more target positions of at least one of an imaging component or a treatment component of a device. In some embodiments, the device may include a rotation base and a pedestal. The operation may further include controlling the at least one of the imaging component or the treatment component to rotate around at least one of a first rotation axis, a second rotation axis, and a third rotation axis based on the one or more target positions; and controlling the imaging component to perform imaging or the treatment component to generate radiotherapy rays. In some embodiments, the first rotation axis may be of the rotation base, the second rotation axis may be parallel with the pedestal and the rotation base, and the third rotation axis may be perpendicular to a surface of the pedestal

Another aspect of the present disclosure provides a non-transitory computer readable medium. The non-transitory computer readable medium may include a set of instructions. When the set of instructions are executed by at least one processor, the set of instructions may direct the at least one processor to perform the following acts. The acts may include determining one or more target positions of at least one of an imaging component or a treatment component of a device. In some embodiments, the device may include a rotation base and a pedestal. The acts may further include controlling the at least one of the imaging component or the treatment component to rotate around at least one of a first rotation axis, a second rotation axis, and a third rotation axis based on the one or more target positions; and controlling the imaging component to perform imaging or the treatment component to generate radiotherapy rays. In some embodiments, the first rotation axis may be of the rotation base, the second rotation axis may be parallel with the pedestal and the rotation base, and the third rotation axis may be perpendicular to a surface of the pedestal.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. The drawings are not to scale. These embodiments are non-limiting exemplary embodiments, in which like reference numerals represent similar structures throughout the several views of the drawings, and wherein:

FIG. 1A is a schematic structural diagram illustrating an exemplary radiation assembly of a medical device according to some embodiments of the present disclosure;

FIG. 1B is a schematic structural diagram illustrating an exemplary radiation assembly of a medical device according to some embodiments of the present disclosure;

FIG. 2 is a schematic structural diagram illustrating an exemplary radiation assembly of a medical device according to some embodiments of the present disclosure;

FIG. 3 is a schematic structural diagram illustrating an exemplary radiation assembly of a medical device according to some embodiments of the present disclosure;

FIG. 4 is a schematic structural diagram illustrating an exemplary radiation assembly for a medical device according to some embodiments of the present disclosure.

FIG. 5A is a schematic structural diagram illustrating an exemplary radiation assembly of a medical device according to some embodiments of the present disclosure;

FIG. 5B is a schematic structural diagram illustrating an exemplary radiation assembly of a medical device according to some embodiments of the present disclosure;

FIG. 5C is a schematic structural diagram illustrating an exemplary radiation assembly of a medical device according to some embodiments of the present disclosure;

FIG. 6 is a schematic structural diagram illustrating an exemplary radiation assembly of a medical device according to some embodiments of the present disclosure;

FIG. 7 is a schematic structural diagram illustrating an exemplary radiation assembly of a medical device according to some embodiments of the present disclosure;

FIG. 8A is a schematic structural diagram illustrating an exemplary radiation assembly of a medical device according to some embodiments of the present disclosure;

FIG. 8B is a schematic structural diagram illustrating an exemplary radiation assembly of a medical device according to some embodiments of the present disclosure;

FIG. 8C is a schematic structural diagram illustrating an exemplary radiation assembly of a medical device according to some other embodiments of the present disclosure;

FIG. 9 is a front view of an exemplary radiation assembly of a medical device according to some embodiments of the present disclosure;

FIG. 10 is a schematic structural diagram illustrating an exemplary medical device according to some embodiments of the present disclosure; and

FIG. 11 is an exemplary flowchart of a method for controlling a medical device according to some embodiments of the present disclosure.

Wherein, the reference signs are: 1—medical device; 10—radiation assembly; 20—housing; 21—opening; 22—accommodation cylinder; 100—pedestal; 110—stator; 111—circular guide rail; 112—arc guide rail; 120—rotor; 121—circular slider; 122—support platform; 1221—annular part; 1222—protruding part; 123—sliding block; 200—rotation base; 210—first rocker arm; 220—second rocker arm; 230—rotation axis; 240—turntable; 250—base; 300—imaging component; 310—imaging radiation source; 320—imaging detector; 400—treatment component; 410—treatment radiation source; 420—treatment detector; 500—rotation component; 510—first support unit; 520—second support unit; 530—bearing seat; 600—third drive component; 610—motor; 620—conveyor; 630—toothed gear transmission mechanism; 631—driving wheel; 632—driven wheel; 633—teeth; 700—first drive component; 710—pneumatic pusher; 720—drive motor; 730—cam transmission structure; 731—cam; 732—driven rod; 200—rotation base; A1—first rotation axis; A2—second radiation axis; A3—third rotation axis.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high-level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

The flowcharts used in the present disclosure illustrate operations that systems implement according to some embodiments of the present disclosure. It is to be expressly understood the operations of the flowcharts may be implemented not in order. Conversely, the operations may be implemented in an inverted order, or simultaneously. Moreover, one or more other operations may be added to the flowcharts. One or more operations may be removed from the flowcharts.

Some embodiments of the present disclosure provide a radiation assembly for a medical device. The radiation assembly may include a diagnostic and treatment component for performing irradiation with radiation that easily passes through a human body and imaging or treating tissue of the human body. The radiation assembly is used in the diagnosis, treatment, and monitoring of diseases, which needs high comprehensiveness and accuracy of irradiation on human tissues.

According to the radiation assembly in some embodiments of the present disclosure, the imaging component and the treatment component may be arranged on the same side (e.g., the same end surface) of the rotation base. When the rotation base rotates around a first rotation axis, the imaging component may scan a patient's body to obtain an image of the patient's body and locate a lesion in the patient's body, and then the treatment component may treat the lesion in the patient's, the whole operation process of which is simple and convenient, speeding up the treatment efficiency. Moreover, the pedestal of the radiation assembly may drive the rotation base to rotate around a third rotation axis, which can enlarge the scanning angle of the imaging component and the treatment component to make the image of the lesion more comprehensive, thereby the lesion may be treated more thoroughly, and the probability of patient recurrence may be reduced. By rotating a pedestal around the first rotation axis and the pedestal around the third rotation axis, the treatment and scanning range of the imaging component and the treatment component may be expanded to a non-coplanar multi-angle treatment and scanning range, making the patient's lesion receives a higher radiation dose, while the surrounding normal tissues receive a lower radiation dose, which improves the positioning accuracy of the lesion, thereby protecting the normal tissues of the human body and improving the safety for radiation delivery. The imaging component and the treatment component may irradiate all sides of the lesion through the non-coplanar multi-angle treatment and scanning without moving the patient, thereby shortening the treatment time and improving the treatment efficiency.

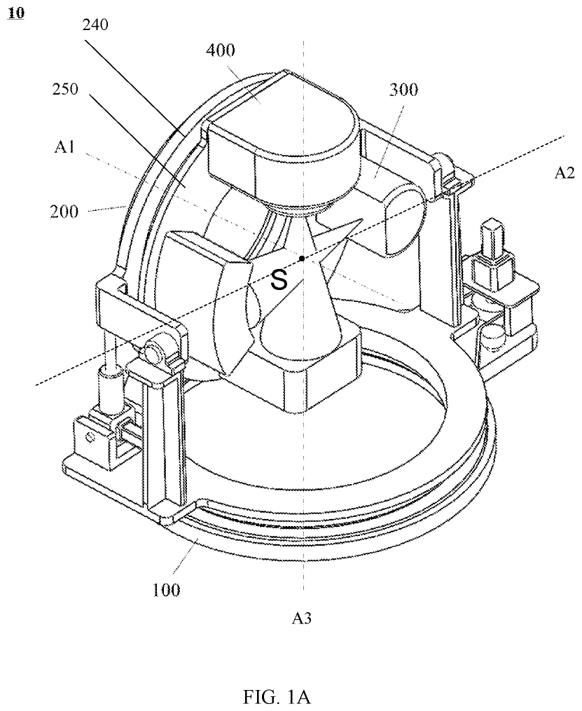

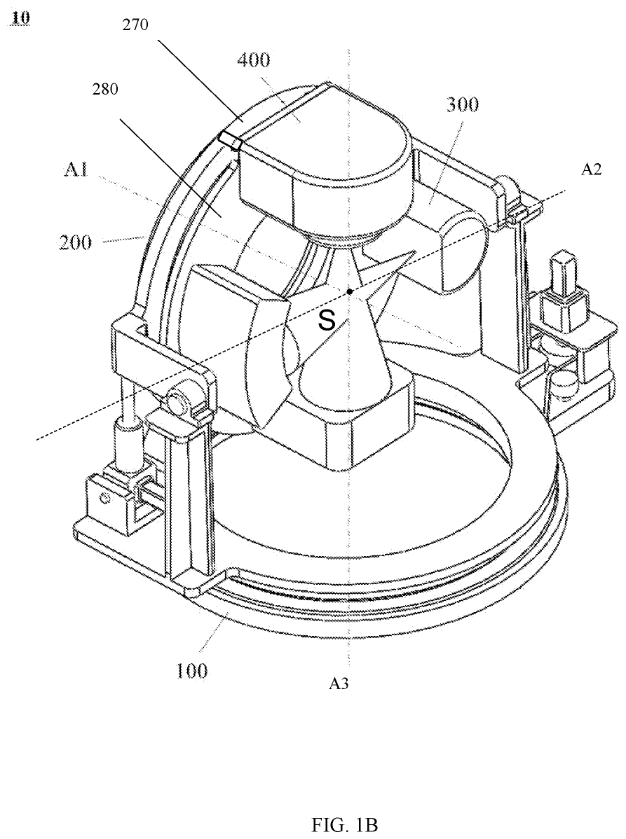

FIG. 1A and FIG. 1B are schematic structural diagrams illustrating an exemplary radiation assembly of a medical device according to some embodiments of the present disclosure. In some embodiments, the radiation assembly 10 may be at a first position, for example, as shown in FIG. 1A. The first position refers to a posture in which the radiation assembly 10 is at an initial position, for example, a position in which the radiation assembly 10 is not working or a position in which a component (e.g., an imaging component and a treatment component) does not rotate any angle around a rotation axis (e.g., a first rotation axis, a second rotation axis, a third rotation axis). In some embodiments, the radiation assembly 10 may also be referred to as a device for brevity.

As shown in FIG. 1A, the radiation assembly 10 may include a pedestal 100, a rotation base 200, an imaging component 300, and a treatment component 400. The rotation base 200 may be set (e.g., disposed) on the pedestal 100, and the imaging component 300 and the treatment component 400 may be set (e.g., disposed) on the rotation base 200. The radiation assembly 10 may emit various radiations, achieving the purpose of imaging or treatment by passing through the human body.

In some embodiments, the imaging component 300 and the treatment component 400 may be arranged on the same side (e.g., the same end surface) of the rotation base 200. In some embodiments, the rotation base 200 may include a rotation unit 250 and a base body 240. The rotation unit 250 may include a turntable, a rotation shaft, etc. The imaging component 300 and the treatment component 400 may be arranged on the rotation unit. The rotation unit may rotate relative to the base body. In some embodiments, the imaging component 300 and the treatment component 400 may have the same isocenter (denoted by point S as shown in FIG. 1A). The isocenter may be an intersection of an axis of the rotation base 200 and an axis of the pedestal 100. In other words, the isocenter of the imaging component 300 and the isocenter of the treatment component 400 may be at the same position. The imaging component 300 and the treatment component 400 may rotate around an axis of the rotation base 200. The axis of the rotation base 200 may also be referred to as the first rotation axis A1. The first rotation axis A1 may be perpendicular to a surface of the rotation base 200 and located at (e.g., pass through) the geometric center of the rotation base 200. The surface of the rotation base 200 may be or be parallel with a surface where the imaging component 300 and the treatment component 400 are located.

In some embodiments, the imaging component 300 and the treatment component 400 may be configured to rotate around the first rotation axis A1 and a second rotation axis A2. The second rotation axis A2 may be parallel with the surface of the rotation base 200.

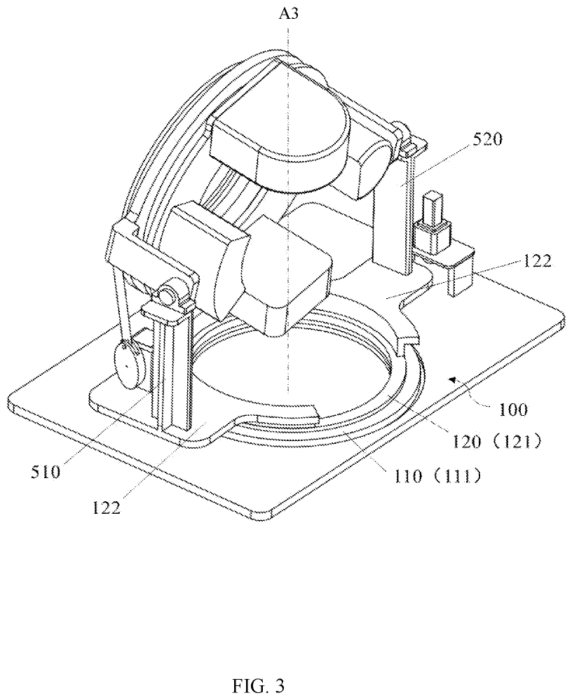

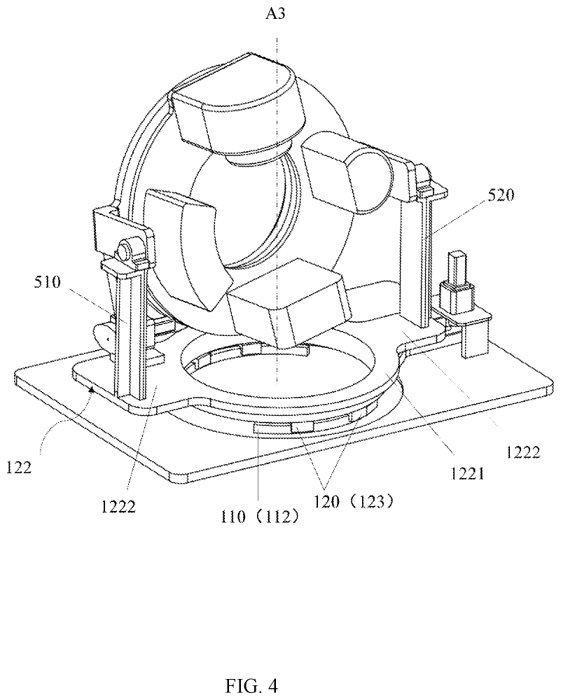

In some embodiments, the pedestal 100 may be configured to rotate around a third rotation axis A3. The imaging component 300 and the treatment component 400 may be configured to rotate around the third rotation axis A3 along with the rotation of the pedestal 100. The third rotation axis A3 may also be referred to as the axis of the pedestal. The third rotation axis A3 may be perpendicular to the bottom surface of the pedestal 100. In some embodiments, the imaging component 300 and the treatment component 400 may be configured to rotate around the first rotation axis A1 and the third rotation axis A3. More descriptions for rotation around the third rotation axis A3 may be found elsewhere in the present disclosure (e.g., FIGS. 3 and 4, and descriptions thereof). For example, the pedestal 100 may include a stator 110 and a rotor 120. The stator 110 refers to a component that does not move relative to the ground, and may be arranged on the ground or other platform. The rotor 120 refers to a component that rotates relative to the stator 110, for example, a component that rotates relative to the stator 110 around the third rotation axis A3.

In some embodiments, the imaging component 300 and the treatment component 400 may be configured to rotate around the first rotation axis A1, the second rotation axis A2, and the third rotation axis A3.

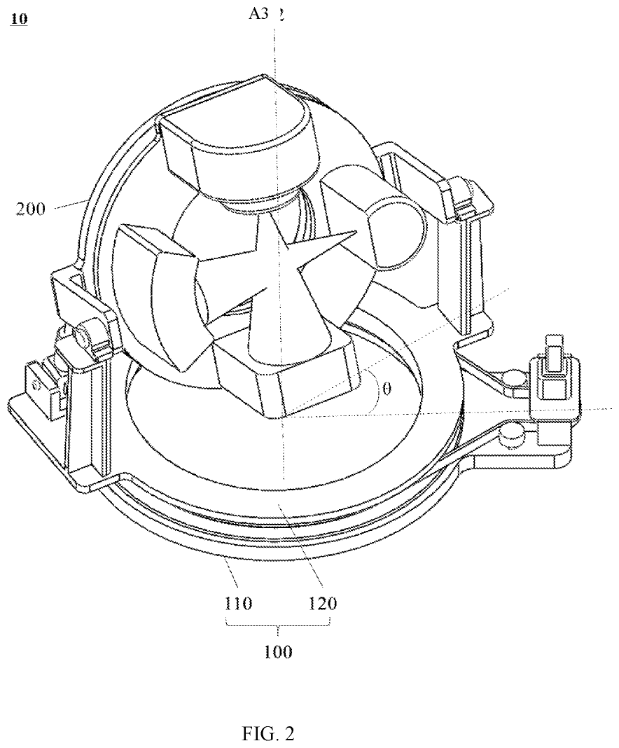

In some embodiments, the pedestal 100 may be configured to provide support for the rotation base 200. The pedestal 100 may include a ring base, a disk base, a frame structure, etc. In some embodiments, the pedestal 100 may rotate around the third rotation axis A3. The third rotation axis A3 may pass through the center of the pedestal 100 and be perpendicular to the bottom surface of the pedestal 100. The center of the pedestal 100 may include the geometric center, the circle center, the gravity center, etc., of the pedestal 100. More descriptions for the rotation of the pedestal 100 around the third rotation axis A3 may be found in FIG. 2.

In some embodiments, the rotation base 200 may include a ring base, a disk base, etc. In some embodiments, the imaging component 300 and the treatment component 400 may be disposed on the same side (e.g., the same end surface) of the rotation base 200. The end surface of the rotation base 200 refers to a surface parallel to the rotation plane of the rotation base 200. The rotation plane refers to a plane where the imaging component 300 and the treatment component 400 rotate. In some embodiments, the rotation base 200 may rotate around the first rotation axis A1. The first rotation axis A1 may pass through the center of the rotation base 200 and be perpendicular to the end surface of the rotation base 200.

In some embodiments, the imaging component 300 may include a medical imaging device that utilizes radiation to irradiate a subject (e.g., a human tissue) for imaging. The imaging component 300 may include a computerized tomography (CT) device, a direct digital radiography (DR) device, a computed radiography (CR) device, etc.

In some embodiments, the treatment component 400 may include a medical instrument that utilizes radiographic transillumination of human body components to achieve therapeutic purposes. The treatment component 400 may include a radiotherapy (RT) device, a nuclear magnetic simulator, etc.

In some embodiments, the imaging component 300 and the treatment component 400 may rotate synchronously along with the rotation of the rotation base 200. When the rotation base 200 rotates around the first rotation axis A1, the imaging component 300 may scan a patient's body for imaging and locating a lesion in the patient's body, and the lesion may be treated by the treatment component 400. The whole operation process may be simple and convenient, improving the treatment efficiency. Moreover, the pedestal 100 of the radiation assembly 10 may drive the rotation base 200 to rotate around the third rotation axis A3, which can enlarge the scanning angle of the imaging component 300 and the treatment angle of the treatment component 400, such that the image of the lesion may be more comprehensive, and the treatment of the lesion may be more thorough, reducing the probability of recurrence of the patient. By rotating the rotation base 200 around the first rotation axis A1 and the pedestal 100 around the third rotation axis A3, the scanning range of the imaging component 300 and the treatment range of the treatment component 400 may be expanded to be non-coplanar and multi-angle, making the patient's lesion receive a higher radiation dose, while the surrounding normal tissue of the lesion receives a lower radiation dose, which improves the positioning accuracy of the lesion, thereby protecting the normal tissue of the human body. Moreover, the imaging component 300 and the treatment component 400 may irradiate all sides of the lesion through the non-coplanar and multi-angle treatment and scanning without moving the patient, thereby shortening the treatment time and improving the treatment efficiency.

FIG. 2 is a schematic structural diagram illustrating the radiation assembly for a medical device as shown in FIG. 1A according to some embodiments of the present disclosure. The radiation assembly 10 is at a second position, as shown in FIG. 2. The second position refers to a posture of the pedestal 100 after the pedestal 100 rotates around the third rotation axis A3.

As shown in FIG. 2, the pedestal 100 may include a stator 110 and a rotor 120. The stator 110 may be disposed on a platform such as the ground. The rotor 120 may rotate relative to (or be rotatable with respect to) the stator 110 and around the third rotation axis A3. The rotation base 200 may be disposed on the rotor 120 and rotate around the third rotation axis A3 together with the rotor 120. For more descriptions regarding the pedestal 100, please refer to FIGS. 3 and 4 and relevant descriptions thereof.

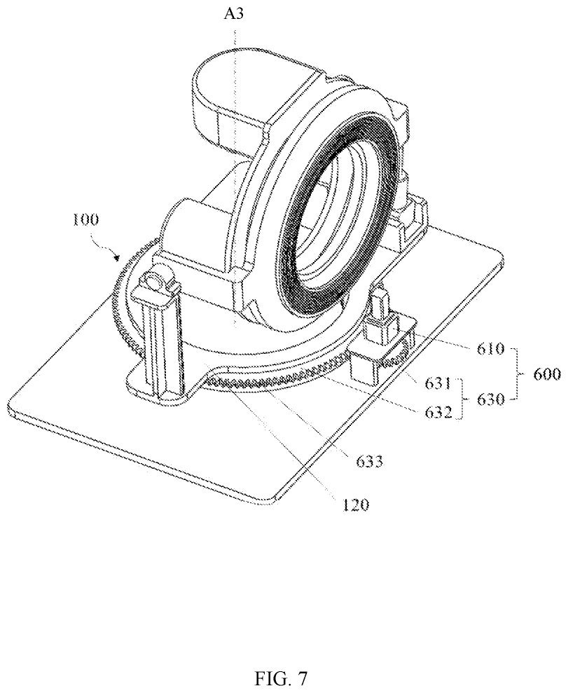

In some embodiments, the rotation angle θ of the pedestal 100 around the third rotation axis A3 may not exceed 90°. In some embodiments, the rotation angle θ of the rotor 120 around the third rotation axis A3 relative to the stator 110 may not exceed 90°. The rotation angle θ of the pedestal 100 may be a rotation angle of the pedestal 100 rotating from the first position to the second position. In some embodiments, the rotation angle θ of the pedestal 100 around the third rotation axis A3 along a clockwise rotation may not exceed 90°. The pedestal 100 may rotate around the third rotation axis A3 along a counterclockwise rotation direction. In some embodiments, the rotation angle θ of the pedestal 100 around the third rotation axis A3 along a counterclockwise rotation direction may not exceed 90°. In some embodiments, the rotation angle θ of the pedestal 100 around the third rotation axis A3 along the clockwise rotation direction may not exceed 45°. In some embodiments, the rotation angle θ of the pedestal 100 around the third rotation axis A3 along the counterclockwise rotation direction may not exceed 45°. In some embodiments, the rotation angle θ of the pedestal 100 around the third rotation axis A3 may be less than or equal to 120°. In application scenarios of some embodiments, structures such as an accommodation cylinder (e.g., an accommodation cylinder 22 as shown in FIG. 7) may be disposed in a radiation range of the radiation assembly 10 for accommodating or passing a couch or the patient. By controlling the rotation angle θ around the third rotation axis A3 not exceeding 90°, the pedestal 100 may be prevented from colliding with the couch or the accommodation cylinder due to its excessive rotation angle. In some embodiments, the accommodation cylinder 22 may include a circular cylinder structure, a polygonal cylinder structure, a special-shaped cylinder structure, etc. In application scenarios of some embodiments, when there are no other components within the radiation range of the radiation assembly 10, the rotation angle θ of the pedestal 100 around the third rotation axis A3 may be controlled to not exceed 90°, which is sufficient to meet the treatment needs of the patient, and avoids the pedestal 100 from colliding with the patient due to its excessive rotation angle.

FIG. 3 is a schematic structural diagram illustrating an exemplary radiation assembly of a medical device according to some embodiments of the present disclosure. FIG. 4 is a schematic structural diagram illustrating an exemplary radiation assembly for a medical device according to some embodiments of the present disclosure.

In some embodiments, the pedestal 100 may include a stator 110 and a rotor 120. The stator 110 refers to a component that does not move relative to the ground, and may be arranged on the ground or other platform. The rotor 120 refers to a component that rotates relative to the stator 110, for example, a component that rotates relative to the stator 110 around the third rotation axis A3.

As shown in FIG. 3, the stator 110 may include a circular guide rail 111. The rotor 120 may include a circular slider 121. The circular guide rail 111 may be rotatably matched with the circular slider 121. For example, the circular slider 121 may be slidably fitted with an inner edge of the circular guide rail 111, or the circular slider 121 may be slidably fitted with the upper surface of the circular guide rail 111.

In some embodiments, the circular slider 121 may be further provided with a support platform 122 for supporting the rotation base 200 and other structures (e.g., a drive component mentioned later, etc.). In some embodiments, the support platform 122 may be designed in a shape according to a position of a component to be supported. For example, the support platform 122 may be configured to support a first support unit (e.g., a first support column) 510 and a second support unit (e.g., a second support column) 520, and the support platform 122 may be designed as two sub-platforms. The two sub-platforms may be spaced and fixed on the circular slider 121, respectively. The first support unit 510 may be fixed on one sub-platform of the two sub-platforms, and the second support unit 520 may be fixed on the other sub-platform of the two sub-platforms.

As shown in FIG. 4, the stator 110 may be composed of a plurality of arc-shaped guide rails 112. The circle center of the plurality of arc-shaped guide rails 112 may be located at the position where the third rotation axis A3 passes through. The rotor 120 may include a plurality of sliding blocks 123. The plurality of sliding blocks 123 may be slidably fitted with the plurality of arc-shaped guide rails 112. In some embodiments, each arc guide rail among the plurality of arc-shaped guide rails 112 may correspond to one or more sliding blocks among the plurality of sliding blocks 123. In some embodiments, a sliding block 123 may be designed as an n-shaped structure, and the sliding block 123 may be slidably sleeved on an arc-shaped guide rail 112.

In some embodiments, the circular slider 121 may be further provided with a support platform 122 for supporting the rotation base 200 and other structures (e.g., the drive component mentioned later, etc.). In some embodiments, the support platform 122 may be designed in a shape according to a position of a component to be supported. For example, the support platform 122 may be configured to support a first support unit 510 and a second support unit 520. The first support unit 510 and the second support unit 520 may be configured to provide a support for the rotation base, the imaging component, and/or the treatment component. The support platform 122 may include an annular part 1221 and two protruding parts 1222. The sliding blocks 123 may be fixed on the bottom surface of the annular part 1221 at intervals. The annular part 1221 may rotate around the third rotation axis A3 relative to the arc-shaped guide rail 112 by the sliding blocks 123. The two protruding parts 1222 may be arranged on two sides of the annular part 1221, respectively. The first support unit 510 may be fixed on one of the two protruding parts 1222, and the second support unit 520 may be fixed on the other one of the two protruding parts 1222.

It should be noted that the two protruding parts 1222 are merely provided for illustration. In some embodiments, the support platform 122 may not include the two protruding parts 1222, and the support platform 122 may include the annular part. The first support unit 510 and the second support unit 520 may be provided on the annular part.

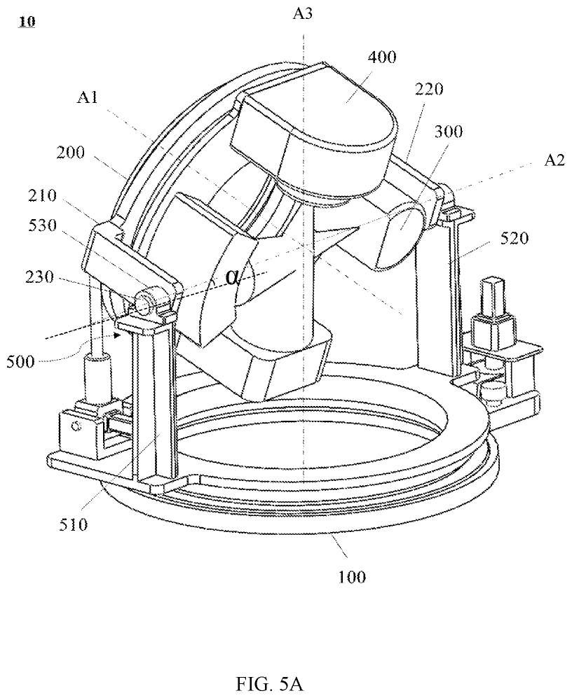

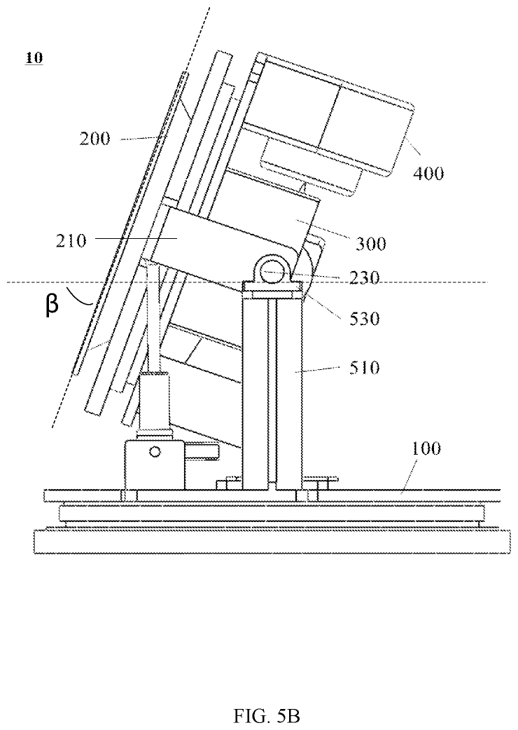

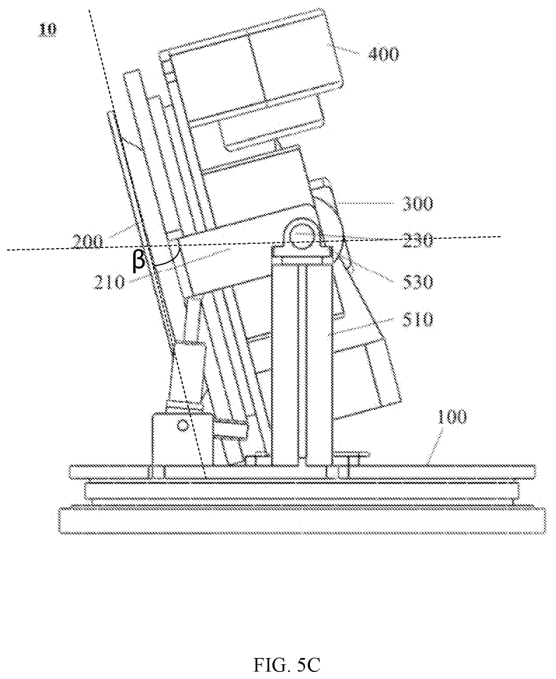

FIGS. 5A to 5C are schematic structural diagrams illustrating an exemplary radiation assembly of a medical device according to some embodiments of the present disclosure. FIG. 5A illustrates a stereo structure of the radiation assembly at a third position, FIG. 5B illustrates a side view of the radiation assembly at the third position, and FIG. 5C illustrates a side view of the radiation assembly at a fourth position. The third position refers to a posture in which the rotation base 200 rotates around a second rotation axis A2 towards the side (e.g., the end surface) where the imaging component 300 and the treatment component 400 are located, and the fourth position refers to a posture in which the rotation base 200 rotates around the second rotation axis A2 towards a side opposite to the side where the imaging component 300 and the treatment component 400 are located.

As shown in FIG. 5A, the radiation assembly 10 may include a rotation component 500 disposed between the pedestal 100 and the rotation base 200. The rotation base 200 may be tilted relative to the pedestal 100 and rotate around the second rotation axis A2 by the rotation component 500. An angle (denoted by a as shown in FIG. 5A) between the second rotation axis A2 and a surface (e.g., the bottom surface) of the pedestal 100 may range from 0° to 5°. For example, the angle between the second rotation axis A2 and the bottom surface of the pedestal 100 may include 0°, 1°, 2°, 3°, 4°, 5°, etc. In some embodiments, the angle between the second rotation axis A2 and the surface (e.g., the bottom surface) of the pedestal 100 may range from 0° to 10°. In some embodiments, the angle between the second rotation axis A2 and the surface (e.g., the bottom surface) of the pedestal 100 may range from 5° to 10°. In some embodiments, the angle between the second rotation axis A2 and the surface (e.g., the bottom surface) of the pedestal 100 may range from 0° to 20°. By setting the rotation base 200 be tilting relative to the pedestal 100 with a certain angle, an error for assembly of the radiation assembly is allowable, which is convenient for the rotation of the imaging component and the treatment component around the second rotation axis A2.

In some embodiments, the angle between the second rotation axis A2 and the surface (e.g., the bottom surface) of the pedestal 100 may range from 0° to 45°. In some embodiments, the angle between the second rotation axis A2 and the surface (e.g., the bottom surface) of the pedestal 100 may range from 0° to 90°. By setting the rotation base 200 be tilting relative to the pedestal 100 with a certain angle, the scanning angle of the imaging component and the treatment angle of the treatment component may be increased, such that the image of the lesion may be more comprehensive, and the treatment of the lesion may be more thorough, thereby reducing the probability of recurrence of the patient.

In some embodiments, the angle between the second rotation axis A2 and the surface (e.g., the bottom surface) of the pedestal 100 may be adjusted based on the rotation component 500. For example, the rotation component 500 may include a first support unit 510 and a second support unit 520 that are arranged on the pedestal 100. The height of at least one of the first support unit 510 and the second support unit 520 may be adjusted. If the height of at least one of the first support unit 510 and the height of the second support unit 520 are different, the angle between the second rotation axis A2 and the surface (e.g., the bottom surface) of the pedestal 100 may be formed. The difference between the height of at least one of the first support unit 510 and the height of the second support unit 520 is, the greater the angle between the second rotation axis A2 and the surface (e.g., the bottom surface) of the pedestal 100 may be. In some embodiments, at least one of the first support unit 510 and the second support unit 520 may include a telescopic column. In some embodiments, at least one of the first support unit 510 and the second support unit 520 may include a transmission mechanism configured to drive the telescopic column to retract. The transmission mechanism may include a gear drive mechanism, a worm gear and worm drive mechanism, a belt drive mechanism, or the like, or a combination thereof.

In some embodiments, the second rotation axis A2 may be parallel with the pedestal 100 (e.g., parallel with the bottom surface of the pedestal 100). That is, the angle between the second rotation axis A2 and the bottom surface of the pedestal 100 may be 0°, or the second rotation axis A2 may be perpendicular to the third rotation axis A3. In some embodiments, the second rotation axis A2 may be substantially parallel with the bottom surface of the pedestal 100. That is, the angle between the second rotation axis A2 and the bottom surface of the pedestal 100 may be greater than 0° and less than or equal to 5°. By rotating the rotation component 500, the rotation base 200 may be tilted relative to the pedestal 100 and rotate around the second rotation axis A2, which may increase the scanning angle of the imaging component 300 and the treatment angle of the treatment component 400, such that the image of the lesion may be more comprehensive, and the treatment of the lesion may be more thorough, thereby reducing the probability of recurrence of the patient. In some embodiments, the second rotation axis A2 may be parallel with (or substantially parallel with) the rotation base 200.

In some embodiments, as shown in FIG. 5B, the rotation base 200 may tilt relative to the pedestal 100 and rotate clockwise around the second rotation axis A2. That is, the rotation base 200 may rotate around the second radiation axis A2 towards the side where the imaging component 300 and the treatment component 400 are located. In some embodiments, as shown in FIG. 5C, the rotation base 200 may tilt relative to the pedestal 100 and rotate counterclockwise about the second rotation axis A2. That is, the rotation base 200 can rotate around the second rotation axis A2 towards the side where the imaging component 300 and the treatment component 400 are not disposed (e.g., towards an opposite side of the side where the imaging component 300 and the treatment component 400 are disposed).

In some embodiments, the rotation base 200 may be tilted relative to the pedestal 100 and rotate clockwise and/or counterclockwise about the second rotation axis A2. In some embodiments, a rotation angle (denoted by angle β as shown in FIGS. 5B and 5C) of the rotation base 200 rotating, by the rotation component 500, around the second rotation axis may be in a range from 5° to 60°. In some embodiments, the rotation angle of the rotation base 200 rotating, by the rotation component 500, around the second rotation axis may be in a range from 1° to 90°. In some embodiments, the rotation angle of the rotation base 200 rotating, by the rotation component 500, around the second rotation axis may be in a range from 5° to 45°. For example, the range may include 10°-30°. As another example, the rotation angle of the rotation base 200 rotating, by the rotation component 500, around the second rotation axis may be in a range from 15° to 25°.

In some embodiments, a rotation angle of the rotation base 200 rotating clockwise around the second rotation axis may be in a range from 5° to 60°. In some embodiments, the rotation angle of the rotation base 200 rotating clockwise around the second rotation axis may be in a range from 5° to 45°. In some embodiments, the rotation angle of the rotation base 200 rotating clockwise around the second rotation axis may be in a range from 15° to 25°.

In some embodiments, a rotation angle of the rotation base 200 rotating counterclockwise around the second rotation axis may be in a range from 5° to 60°. In some embodiments, the rotation angle of the rotation base 200 rotating counterclockwise around the second rotation axis may be in a range from 5° to 45°. In some embodiments, the rotation angle of the rotation base 200 rotating counterclockwise around the second rotation axis may be in a range from 15° to 25°.

As shown in FIG. 5A, in some embodiments, the rotation component 500 may include the first support unit 510 and the second support unit 520 that are arranged on the pedestal 100. The rotation base 200 may be connected with a first rocker arm 210 and a second rocker arm 220. The first rocker arm 210 may be rotatably connected with the first support unit 510. That is, the first rocker arm 210 may be connected with the first support unit 510 and be ratable around the second rotation axis A2. The second rocker arm 220 may be rotatably connected with the second support unit 520. That is, the second rocker arm 220 may be connected with the second support unit 520 and be rotatable around the second rotation axis A2. In some embodiments, the rotatable connection may include a hinge connection, a pivot connection, etc.

In some embodiments, the rotation component 500 may include other structures, which are not limited herein. For example, the rotation component 500 may include two triangular brackets arranged on the pedestal 100, and the two triangular brackets may be rotatably connected with the two rocker arms, respectively.

In some embodiments, each of the top ends of the first support unit 510 and the second support unit 520 connected with the first rocker arm 210 and the second rocker arm 220 may be provided with a bearing seat 530. As used herein, the top end of the first support unit 510 or the second support unit 520 refers to an end of the first support unit 510 or the second support unit 520 that is far away from the pedestal 100. Ends of the first rocker arm 210 and the second rocker arm 220 (e.g., ends of the first rocker arm 210 and the second rocker arm 220 that are close to the first support unit 510 and the second support unit 520, respectively) may be provided with a rotation axis 230. The rotation axis 230 may be rotatable and inserted in the bearing seat 530, such that the rocker arms (e.g., the first rocker arm 210 and the second rocker arm 220) and the support units (e.g., the first support unit 510 and the second support unit 520) may be rotatably connected.

In some embodiments, the first support unit 510 and the second support unit 520 may include columns arranged perpendicular to the pedestal 100. Cross-sections of the first support unit 510 and the second support unit 520 perpendicular to axial directions of the first support unit 510 and the second support unit 520 may be configured as a T-shape for improving support strength. In some embodiments, the cross-sections of the first support unit 510 and the second support unit 520 may be in other shapes, such as ellipse, rectangle, and polygon.

In some embodiments, at least one of the first rocker arm 210 and the second rocker arm 220 may be in a plate shape and be perpendicular to the rotation base 200. The first rocker arm 210 and the second rocker arm 220 may be arranged on the outer edge of the rotation base 200, respectively, avoiding motion interference with the rotation of the imaging component 300 and the treatment component 400.

In some embodiments, the first support unit 510 and the second support unit 520 may be arranged on the pedestal 100 at an interval of 180°, that is, a line connecting the top ends of the first support unit 510 and the second support unit 520 away from the rotation base 200 may be intersected with the third rotation axis A3. In other words, the second rotation axis A2 may intersect the third rotation axis A3. In this way, when the pedestal 100 rotates around the third rotation axis A3 and the rotation base 200 rotates around the second rotation axis A2, the imaging component 300 and the treatment component 400 may be driven to rotate around an intersection of the second rotation axis A2 and the third rotation axis A3, which is conducive to scanning at various angles centered on the patient's lesion. In some embodiments, the second rotation axis A2 may not intersect with the third rotation axis A3.

In some embodiments, the first rocker arm 210 and the second rocker arm 220 may be arranged at an interval of 180° on the rotation base 200, that is, a line connecting the ends of the first rocker arm 210 and the second rocker arm 220 away from the end of the rotation base 200 may intersect the second rotation axis A2. In other words, the second rotation axis A2 may intersect the first rotation axis A1. In this way, when the rotation base 200 rotates around the second rotation axis A2 and the first rotation axis A1, the imaging component 300 and the treatment component 400 may be driven to rotate around the intersection of the second rotation axis A2 and the first rotation axis A1, which is conducive to scanning at various angles centered on the patient's lesion. In some embodiments, the second rotation axis A2 may not intersect the first rotation axis A1.

In some embodiments, the first rotation axis A1, the third rotation axis A3, and the second rotation axis A2 may intersect at the same isocenter. In some embodiments, the first rotation axis A1, the third rotation axis A3, and the second rotation axis A2 may move around a common center point. A ray may pass through the smallest sphere centered on the common center point, and the common center point may be the isocenter, such that the imaging component 300 and the treatment component 400 may be capable of rotating in three directions based on the isocenter. In application scenarios of some embodiments, a region of the patient to be scanned or treated may be placed on or near the isocenter of the three rotation axes, so as to perform accurate and comprehensive scanning and treatment on the region.

In some embodiments, the imaging component 300 and the treatment component 400 may be capable of rotating in one direction. For example, the imaging component 300 and the treatment component 400 may rotate around one of the first rotation axis A1, the second rotation axis A2, and the third rotation axis A3.

In some embodiments, the imaging component 300 and the treatment component 400 may be capable of rotating in two directions. For example, the imaging component 300 and the treatment component 400 may rotate around the first rotation axis A1 and the second rotation axis A2. As another example, the imaging component 300 and the treatment component 400 may rotate around the first rotation axis A1 and the third rotation axis A3. As still another example, the imaging component 300 and the treatment component 400 may rotate around the second rotation axis A2 and the third rotation axis A3.

In some embodiments, the imaging component 300 and the treatment component 400 may be capable of rotating in three directions. For example, the imaging component 300 and the treatment component 400 may rotate around the first rotation axis A1, the second rotation axis A2, and the third rotation axis A3. The imaging component 300 and the treatment component 400 may rotate around the third rotation axis A3 along with the rotation of pedestal around the third rotation axis A3.

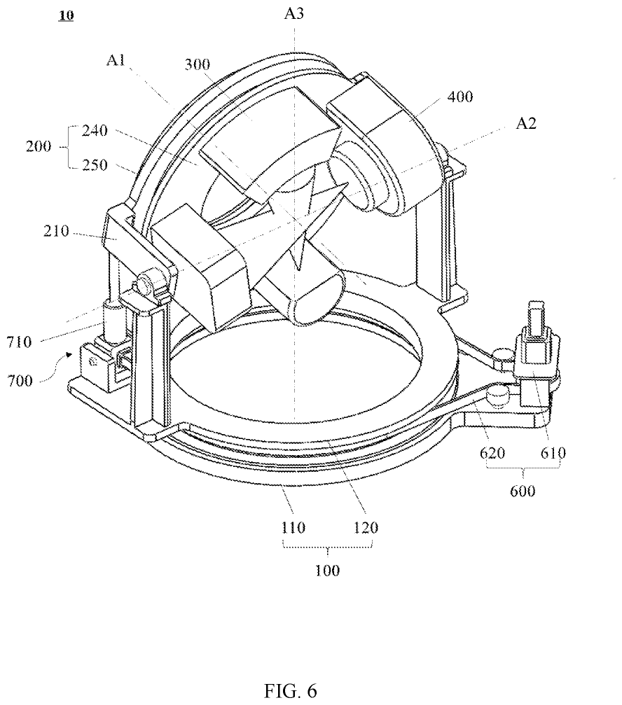

FIG. 6 is a schematic structural diagram illustrating an exemplary radiation assembly of a medical device according to some embodiments of the present disclosure. The radiation assembly 10 is at a fifth position, as shown in FIG. 6. The fifth position refers to a posture of the pedestal 100 after the imaging component 300 and the treatment component 400 rotate around the first rotation axis A1, the pedestal 100 rotates around the third rotation axis A3, and the rotation base 200 rotates around the second rotation axis A2.

As shown in FIG. 6, in application scenarios of some embodiments, according to the structure of the radiation assembly 10, the patient may lie flat within the radiation range of the imaging component 300 and the treatment component 400. At this time, by rotating the rotation base 200 around the first rotation axis A1, the patient may be scanned or treated in a circumferential direction. By rotating the rotation base 200 around the second rotating axis A2, the patient may be scanned or treated in a head-foot direction of the patient. By rotating the pedestal 100 around the third rotation axis A3, the patient may be scanned or treated on the left and right sides of the patient. The imaging component 300 and the treatment component 400 may have three degrees of freedom by rotating the rotation base 200 and the pedestal 100, which may realize a larger scanning range, thereby the scanning range and angle of the patient's brain or body may be more three-dimensional and comprehensive, and the treatment effect may be improved.

In application scenarios of some embodiments, during scanning or treatment of the patient, the imaging component 300 and the treatment component 400 may control the rotation base 200 to rotate around the first rotation axis A1 and/or around the second rotation axis A2, and/or, control the pedestal 100 to rotate around the second rotation axis A2. That is, the imaging component 300 and the treatment component 400 may scan or treat while rotating, such that continuous multi-angle scanning and treatment may be realized.

In application scenarios of some embodiments, during the imaging component 300 and the treatment component 400 scan or treats the patient, the positions of the imaging component 300 and the treatment component 400 may remain unchanged. When the scanning or treatment process stops, the rotation base 200 may be controlled to rotate around the first rotation axis A1 and/or the second rotation axis A2, and/or the pedestal 100 may be controlled to rotate around the third rotation axis A3. When the imaging component 300 and the treatment component 400 are rotated in place, the imaging component 300 and the treatment component 400 may be initiated to perform the scanning or treatment.

In some embodiments, the rotation base 200 may include a rotation unit including a turntable 240 capable of rotating relative to the first rotation axis A1. Both the imaging component 300 and the treatment component 400 may be arranged on the same turntable 240, which can realize the synchronous control of the imaging component 300 and the treatment component 400, and simplify the structure and control strategy.

In some embodiments, the rotation base 200 may include a body body base 250. The body base 250 may include a structure of the rotation base 200 that does not rotate. The turntable 240 of the rotation base 200 may be rotatably disposed on the body base 250. The body base 250 may provide support for the turntable 240. In some embodiments, first rocker arm 210 and second rocker arm 220 (as shown in FIG. 5A) may be fixed to body base 250.

In some embodiments, the imaging component 300 and the treatment component 400 may rotate separately or independently. The imaging component 300 and the treatment component 400 may rotate relative to (or be rotatable with respect to) each other. As shown in FIG. 1B, the rotation base 200 may include a first rotation unit 270 and a second rotation unit 280. The treatment component 400 may be disposed on the first rotation unit 270. The imaging component 300 may be disposed on the second rotation unit 280. The second rotation unit may rotate relative to (or be rotatable with respect to) the first rotation unit. For example, the first rotation unit 270 and the second rotation unit may share the same rotation shaft. In some embodiments, the first rotation unit may include a turntable and rotate along with the turntable around the first rotation axis A1. In some embodiments, the second rotation unit may include a turntable and rotate along with the turntable 240 around the first rotation axis A1. By the first rotation unit and the second rotation unit, the imaging component 300 and the treatment component 400 may be controlled to rotate independently, and an angle between the imaging component 300 and the treatment component 400 may be adjusted, which improves the flexibility of the treatment process. Both the first rotation unit and the second rotation unit may rotate relative to (or be rotatable with respect to) the turntable 240 and around the first rotation axis A1, such that the degrees of freedom of the first rotation unit and the second rotation unit relative to the body base 250 may be increased.

In some embodiments, the radiation assembly 10 may further include a first drive component (not shown in the figure). The first drive component may be configured to drive the rotation base 200 to rotate around the first rotation axis A1. In some embodiments, the first drive component may include a motor and a gear train. The motor may be disposed on the body base 250 of the rotation base 200, and the motor may be connected with the turntable 240 through the gear train. The motor may drive the gear train through forward rotation or reverse rotation, and the gear train may drive the turntable 240 to rotate clockwise or counterclockwise relative to the body base 250 and around the first rotation axis A1. In some embodiments, the first drive component may include other structures.

In some embodiments, the radiation assembly 10 may further include a second drive component 700. The second drive component 700 may be configured to drive the rotation base 200 to rotate around the second rotation axis A2. In some embodiments, the second drive component 700 may include a pneumatic pusher 710. One end of the pneumatic pusher 710 may be connected with the pedestal 100, and the other end of the pneumatic pusher 710 may be connected with the rotation base 200. The pneumatic pusher 710 may drive the rotation base 200 to rotate clockwise or counterclockwise around the second rotation axis A2 by extending or shortening the pneumatic pusher 710. In some embodiments, the first drive component 700 may include a hydraulic pusher, an electric pusher, or other structures.

In some embodiments, there may be one second drive component 700, and the one second drive component 700 may be disposed on any side of the rotation base 200 to simplify the structure. For example, the pneumatic pusher 710 of the second drive component 700 may be arranged close to the first rocker arm 210 or the second rocker arm 220 of the rotation base 200.

In some embodiments, there may be multiple second drive components 700. The multiple second drive components 700 may be arranged around the rotation base 200 at intervals. For example, there may be two second drive components 700, one of which may be disposed on a side close to the first rocker arm 210 the rotation base 200, and the other second drive component 700 may be disposed on a side close to the second rocker arm 220 of the rotation base 200. By synchronously driving the multiple first drive components 700, the rotation base 200 may be rotated around the second rotation axis A2. By the multiple second drive components 700, the rotation stability of the rotation base 200 may be improved.

In some embodiments, the radiation assembly 10 may further include a third drive component 600. The third drive component 600 may be configured to drive the pedestal 100 to rotate around the third rotation axis A3. In some embodiments, the pedestal 100 may include a stator 110 and a rotor 120. The third drive component 600 may include a motor 610 and a conveyor 620. The motor 610 may be connected with the conveyor 620. The conveyor 620 may be wound on the rotor 120. The motor 610 may drive the conveyor 620 to rotate through forward rotation and reverse rotation, and the conveyor 620 may drive the rotor 120 to rotate clockwise or counterclockwise relative to the stator 110 and around the third rotation axis A3. In some embodiments, the third drive component 600 may include other structures.

In some embodiments, the first drive component, the second drive component, and/or the third drive component may be the same. For example, the first drive component, the second drive component, and the third drive component may include a pneumatic pusher. As another example, the first drive component, the second drive component, and the third drive component may include a motor and a conveyor. In some embodiments, at least two of the first drive component, the second drive component, and/or the third drive component may be different. For example, the first drive component may include a pneumatic pusher, and each of the second drive component, and the third drive component may include a motor and a conveyor.

FIG. 7 is a schematic structural diagram illustrating an exemplary radiation assembly of a medical device according to some embodiments of the present disclosure.

As shown in FIG. 7, in some embodiments, the third drive component 600 may include a motor 610 and a toothed gear transmission mechanism 630. In some embodiments, the toothed gear transmission mechanism 630 may include a driving wheel 631 and a driven wheel 632. The motor 610 may be connected with the driving wheel 631. The driving wheel 631 may mesh with the driven wheel 632. The driven wheel 632 may be connected with the rotor 120 of the pedestal 100. The motor 610 may drive the driving wheel 631 to rotate. The driving wheel 631 may drive the driven wheel 632 to rotate. The driven wheel 632 may drive the rotor 120 of the pedestal 100 to rotate around the third rotation axis A3. In some embodiments, the driven wheel 632 may be a separate structure from the rotor 120, and the driven wheel 632 may be fixed on the rotor 120. In some embodiments, the driven wheel 632 may be integrated with the rotor 120, and the outer edge of the rotor 120 may be formed with the teeth 633 meshing with the driving wheel 631, and the driving wheel 631 may drive the rotor 120 to rotate through the teeth 633.

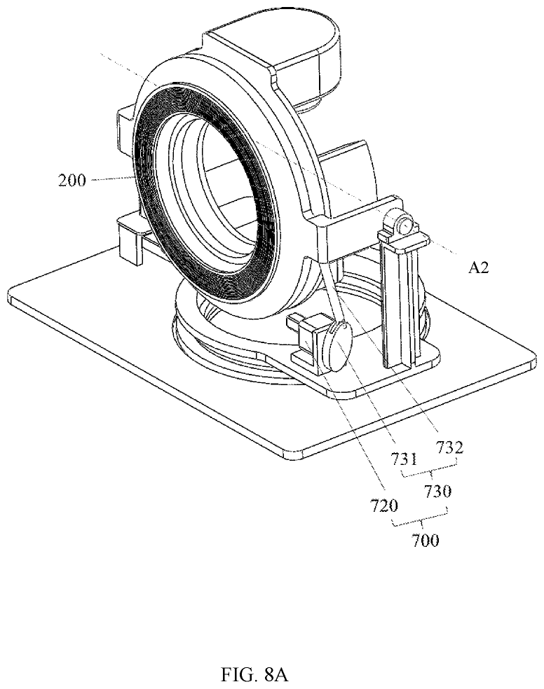

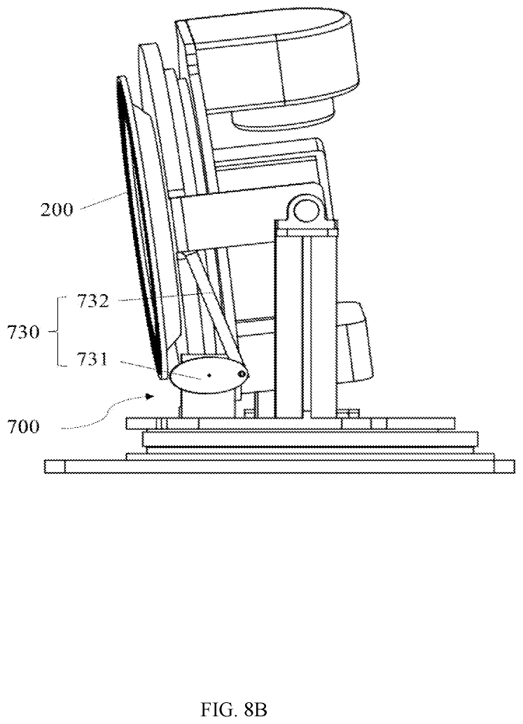

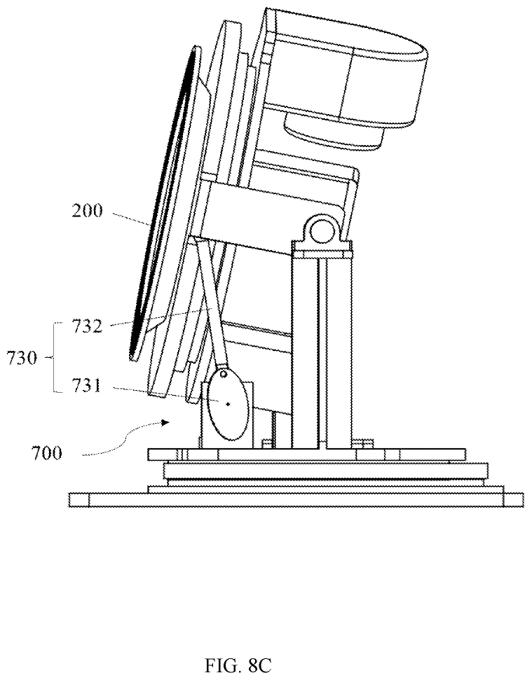

FIGS. 8A to 8C are structural schematic diagrams illustrating an exemplary radiation assembly of a medical device according to some other embodiments of the present disclosure.

As shown in FIGS. 8A to 8C, the first drive component 700 may include a driving motor 720 and a cam transmission structure 730. In some embodiments, the cam transmission structure 730 may include a cam 731 and a driven rod 732. The drive motor 720 may be connected with the cam 731. One end of the cam 731 may be hinged with the driven rod 732, and the other end of the driven rod 732 may be hinged with the rotation base 200. The driving motor 720 may drive the cam 731 to rotate. The cam 731 may drive the driven rod 732 to swing. The driven rod 732 may drive the rotation base 200 to rotate around the second rotation axis A2.

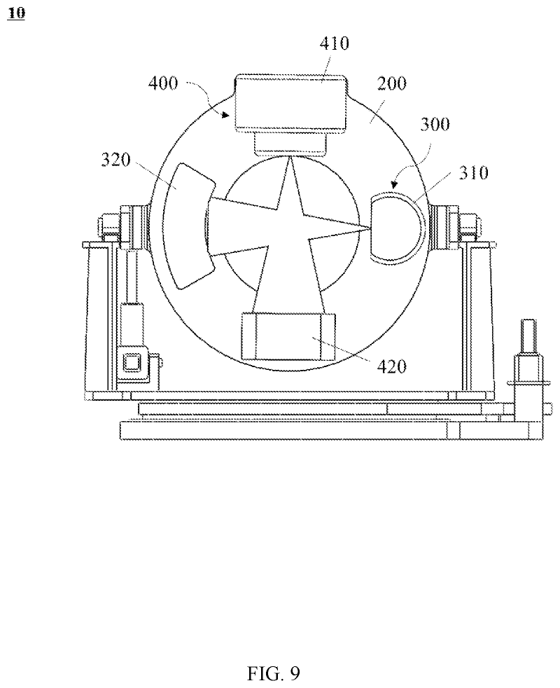

FIG. 9 is a front view of an exemplary radiation assembly of a medical device according to some embodiments of the present disclosure. The radiation assembly is at the first position, as shown in FIG. 9.

As shown in FIG. 9, in some embodiments, the imaging component 300 may include an imaging radiation source 310 and an imaging detector 320. The imaging radiation source 310 may emit X-rays, photon rays, etc. The imaging detector 320 may receive the X-rays and convert the X-rays into electrical signals. The electrical signal may be sent to a device such as a computer or a processor. The device, such as the computer or processor, may develop and convert the scanning result of the imaging component 300 into one or more images.

In some embodiments, the treatment component 400 may include a treatment radiation source 410 and a treatment detector 420. The treatment radiation source 410 may emit rays such as α-rays, β-rays, γ-rays, X-rays, electron beams, photon beams, proton beams, heavy beams, etc. The treatment detector 420 may receive corresponding α-rays, β-rays, γ-rays, etc. The rays from the treatment radiation source 410 may have a therapeutic effect on tumors or lesions after passing through the tumors or lesions of the patient.

In some embodiments, the imaging radiation source 310 and the treatment radiation source 410 may be disposed on the rotation base 200 at intervals. In some embodiments, the imaging radiation source 310 and the treatment radiation source 410 may be arranged at any angular interval. For example, an angle between the imaging radiation source 310 and the treatment radiation source 410 relative to the first rotation axis A1 may include 30°, 45°, 90°, 135°, 150°, etc. In some embodiments, the imaging radiation source 310 and the imaging detector 320 may be arranged at an interval of 180° to facilitate the imaging detector 320 to receive the rays of the imaging radiation source 310. In some embodiments, the treatment radiation source 410 and the treatment detector 420 may be arranged at an interval of 180° to facilitate the treatment detector 420 to receive the rays of the treatment radiation source 410.

In some embodiments, the line connecting the imaging radiation source 310 and the imaging detector 320 may be perpendicular to the line connecting the treatment radiation source 410 and the treatment detector 420. That is, the angle between the imaging radiation source 310 and the treatment radiation source 410 relative to the first rotation axis A1 may be 90°. The arrangement of the imaging radiation source 310 and the treatment rotation source 410 may minimize the ray interference between the imaging radiation source 310 and the treatment radiation source 410.

In some embodiments, the angle between the line connecting the imaging radiation source 310 and the imaging detector 320 and the line connecting the treatment radiation source 410 and the treatment detector 420 may be not 90°, for example, 80 degrees, 100 degrees, etc.

In some embodiments, the rotation base 200 may further be provided with a first motion guide rail (not shown in the figure). The first motion guide rail may be arranged between the imaging component 300 and the rotation base 200, and the imaging component 300 may move relative to the rotation base 200 by the first moving rail.

In some embodiments, the rotation base 200 may further be provided with a second motion guide rail (not shown in the figure). The second motion guide rail may be arranged between the treatment component 400 and the rotation base 200, and the treatment component 400 may move relative to the rotation base 200 by the second motion guide rail.

In some embodiments, only the first motion guide rail or the second motion guide rail may be provided on the rotation base 200. In some embodiments, the first motion guide rail and the second motion guide rail may be simultaneously provided on the rotation base 200.

In some embodiments, the first motion guide rail and/or the second motion guide rail may include an arc shape. The extension direction of the first motion guide rail and/or the second motion guide rail may take the isocenter as the center of a circle corresponding to the arc shape. A plane where the first motion guide rail and/or the second motion guide rail are located may be perpendicular to the surface of the rotation base 200. In some embodiments, the first moving guide rail and/or the second moving guide rail may also have other shapes and be arranged extending along other directions. By setting the first motion guide rail and/or the second motion guide rail, the degree of freedom of the imaging component 300 and/or the treatment component 400 may be increased, which improves the flexibility of scanning.

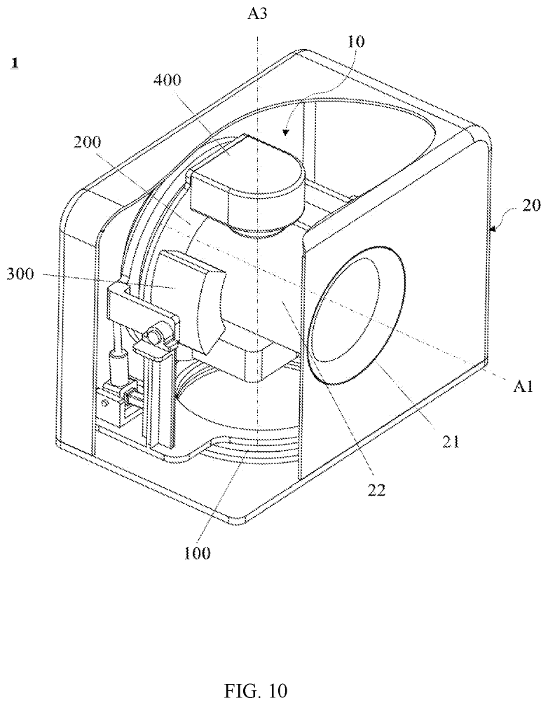

FIG. 10 is a schematic structural diagram illustrating an exemplary medical device according to some embodiments of the present disclosure. A part of a housing 20 is hidden to illustrate the radiation assembly 10 in the medical device.

In some embodiments of the present disclosure, a medical device 1 is provided. The medical device 1 may include the radiation assembly 10 as described elsewhere in the present disclosure.

In some embodiments, the medical device 1 may include a radiation assembly 10. The radiation assembly 10 may include a pedestal 100, a rotation base 200 arranged on the pedestal 100, an imaging component 300, and a treatment component 400. The imaging component 300 and the treatment component 400 may be disposed on the same side of the rotation base 200. The rotation base 200 may rotate along a horizontal plane following the pedestal 100. That is, the rotation base 200 may follow a motion trajectory of the pedestal 100 to rotate, presenting an arc shape in the horizontal plane. In some embodiments, the rotation base 200 can follow the pedestal 100 to rotate around the second rotating axis A2. In some embodiments, the rotation base 200 may follow the pedestal 100 to rotate around other axes, which is not limited in the present disclosure.

In some embodiments, the medical device 1 may further include an accommodation cylinder 22 and the housing 20. The accommodation cylinder 22 may pass through a hole in the rotation base 200 of the radiation assembly 10. The housing 20 may be configured to accommodate the radiation assembly 10. In some embodiments, the pedestal 100 of the radiation assembly 10 may rotate relative to (or be rotatable with respect to) the housing 20 around the third rotation axis A. The third rotation axis A3 may be perpendicular to the bottom surface of the housing 20. The pedestal 100 of the radiation assembly 10 may drive the rotation base 200 to rotate around the third rotation axis A3, which may increase the scanning angle and the treatment angle of the imaging component 300 and the treatment component 400, such that the image of the lesion can be more comprehensive, and the treatment of the lesion can be more thorough, thereby reducing the probability of recurrence of the patient. The housing 20 may accommodate the radiation assembly 10, which can protect and prevent the radiation assembly 10 from dust.

In some embodiments, the housing 20 may be configured as a rectangular hexahedron housing. One side of the housing 20 may have an opening 21 to built-in the accommodation cylinder 22. One end of the accommodation cylinder 22 may be connected with the opening 21 of the housing 20, and the other end of the accommodation cylinder 22 may extend to the radiation range of the imaging component 300 and the treatment component 400, such that a space for treatment and scanning may be formed in the accommodation cylinder 22. In some embodiments, the axis of the accommodation cylinder 22 may coincide with the first rotation axis A1. In some embodiments, both ends of the accommodation cylinder 22 may be connected with the housing 20.

In application scenarios of some embodiments, the accommodation cylinder 22 may allow a couch to go in and out. The patient may lie on the couch and enter the accommodation cylinder 22 through the opening 21 of the housing 20. The imaging component 300 and the treatment component 400 may be activated for scanning and treating the patient.

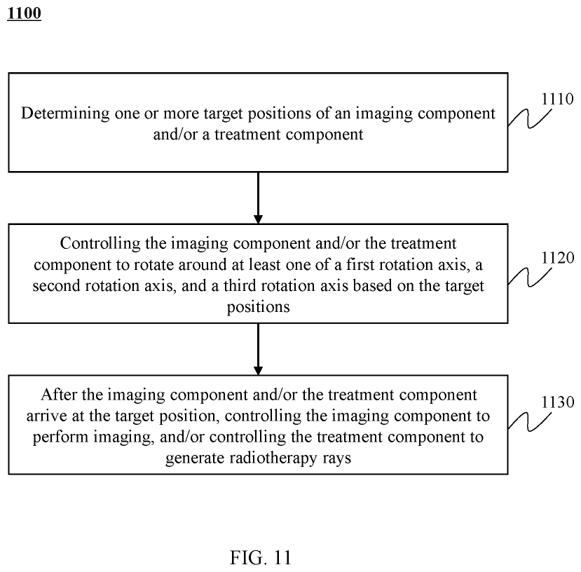

FIG. 11 is a flowchart illustrating an exemplary process for controlling a medical device according to some embodiments of the present disclosure.

According to some embodiments of the present disclosure, a method for controlling a medical device is provided. The method for controlling a medical device may include a process 1100. As shown in FIG. 11, the process 1100 may be executed by a computing device including at least one processor and a storage device, and include the following operations.

In 1110, one or more target positions of the imaging component 300 and/or the treatment component 400 may be determined.

In some embodiments, the target position of the imaging component 300 and/or the treatment component 400 may be position information that is pre-input into the controller and/or processor. For example, the position information may be formulated by medical personnel according to an actual condition of a patient.

In some embodiments, the target positions may include a certain point or a certain area within a motion range of the imaging component 300 and/or the treatment component 400. In some embodiments, the target positions may be determined by a coordinate system with the isocenter as the origin. In some embodiments, a three-dimensional coordinate system may be established with the isocenter as the origin and the first rotation axis A1, the second rotation axis A2, and the third rotation axis A3 as coordinate axes. The target position may be determined based on the three-dimensional coordinate system. In some embodiments, the target positions may also be determined based on a spherical coordinate system.

In some embodiments, one or more rotation parameters may be determined based on personalized data of a subject (e.g., a patient). In some embodiments, the personalized data of the subject may include the size of the subject, the shape of the subject, etc. During imaging, the controller may determine a target position where the imaging component (e.g., the imaging radiation source) needs to be located and determine one or more rotation parameters based on the target position. The one or more rotation parameters may include a rotation direction of the imaging component, a rotation angle of the imaging component, a duration for the rotation angle, a rotation axis (e.g., the first rotation axis, the second rotation axis, the third rotation axis, etc.), etc. For example, the rotation angle of the imaging component around the second rotation axis may be associated with the length of the subject along the direction parallel to the first rotation axis. The greater the length of the subject along the direction parallel with the first rotation axis, the greater the rotation angle of the imaging component around the second rotation axis may be. As another example, the controller may determine one of the first rotation axis, the second rotation axis, and the third rotation axis according to the target position.

In some embodiments, the personalized data may include a radiotherapy plan of the subject. The controller may determine a motion trajectory of the treatment component (e. g., the treatment radiation source) based on the radiotherapy plan. The motion trajectory may include multiple target positions where the treatment component needs to arrive. The controller may determine one or more rotation parameters based on the target positions as described above.

In 1120, the imaging component 300 and/or the treatment component 400 may be controlled to rotate around at least one of the first rotation axis A1, the second rotation axis A2, and the third rotation axis A3 based on the target positions.

In some embodiments, according to the target position, the controller and/or processor may determine a difference between a current position of the imaging component 300 and/or the treatment component 400 and the target position in the three-dimensional coordinate system. The controller and/or processor may determine what axis axes among the first rotation axis A1, the second rotation axis A2, and the third rotation axis A3, the imaging component 300 and/or the treatment component 400 needs to rotate around. The controller and/or processor may further determine specific rotation angle(s) of the axis/axes that the imaging component 300 and/or the treatment component 400 needs to rotate around.

The controller and/or processor may control the imaging component 300 and/or the treatment component 400 to rotate around the determined rotation axis/axes by the corresponding rotation angle(s), such that the imaging component 300 and/or the treatment component 400 may reach the target position.

In 1130, after the imaging component 300 and/or the treatment component 400 arrive at the target position, the imaging component 300 may be controlled to perform imaging, and/or the treatment component 400 may be controlled to generate radiotherapy rays.

In some embodiments, after the imaging component 300 and/or the treatment component 400 arrive at the target position, it means that the radiation of the imaging component 300 and/or the treatment component 400 may be aimed at the lesion region of the patient. At this time, the controller and/or the processer may control the imaging component 300 to start generating radiation to pass through the lesion region for imaging, and/or control the treatment component 400 to start generating radiotherapy rays to pass through the lesion region for treatment.

In some embodiments, when the medical device 1 is at the first position, the rotation base 200 may be controlled to rotate around the first rotation axis A1, such that the imaging component 300 on the rotation base 200 may perform imaging, or the rotation base 200 may be controlled to rotate around the first rotation axis A1 to enable the imaging component 300 on the rotation base 200 to perform imaging, and to enable the treatment component 400 on the rotation base 200 to generate radiotherapy rays.

In some embodiments, the first position of the medical device 1 refers to a posture in which the radiation assembly 10 is at an initial position.

In some embodiments, the controller may control the rotation base 200 to rotate around the first rotation axis A1, such that the imaging component 300 on the rotation base 200 may perform imaging, and the imaging component 300 may send the image of the patient to the computer or the processor for processing.

In some embodiments, the controller may control the rotation base 200 to rotate around the first rotation axis A1, such that the imaging component 300 located on the rotation base 200 may perform imaging, and the treatment component 400 located on the rotation base 200 may perform treatment. That is, after the imaging component 300 obtains the image of the patient, the controller may control the treatment component 400 to generate radiotherapy rays to treat the lesion based on the image of the lesion.

In some embodiments, the controller may control the rotation base 200 to rotate around the first rotation axis A1, and at the same time, activate the imaging component 300 to perform imaging and obtain an image of the lesion of the patient. Further, the controller may control the imaging component 300 to be turned off, and control the treatment component 400 to be turned on, so as to perform treatment based on the image of the patient's lesion.

In some embodiments, the controller may control the rotation base 200 to rotate around the first rotation axis A1, activate the imaging component 300 to perform imaging, and at the same time, activate the treatment component 400 to generate radiotherapy rays for treatment, such that the patient's lesions may be imaged and treated at the same time, improving the treatment efficiency.

In some embodiments, when the medical device 1 is at the first position, pedestal 100 may be controlled to rotate around the third rotation axis A3, such that the treatment component 400 on the rotation base 200 may generate radiotherapy rays; or the rotation base 200 may be controlled to rotate around the second rotation axis A2, such that the treatment component 400 on the rotation base 200 may generate radiotherapy rays. The rotation angle of the pedestal 100 around the third rotation axis A3 may be controlled to be less than 90 degrees.

In some embodiments, the controller may control the pedestal 100 to rotate around the third rotation axis A3, such that the treatment component 400 on the rotation base 200 may generate radiotherapy rays, which enlarges the scanning angle of the radiotherapy rays of the treatment component 400 on the left and right sides of the patient, thereby making the treatment more thorough.

In some embodiments, the controller may control the rotation base 200 to rotate around the second rotation axis A2, such that the treatment component 400 on the rotation base 200 may generate radiotherapy rays, which enlarges the scanning angle of the radiotherapy rays of the treatment component 400 on the patient's head and feet, thereby making the treatment more thorough.

In some embodiments, the controller may control the pedestal 100 to rotate around the third rotation axis A3, and/or control the rotation base 200 to rotate around the second rotation axis A2, and at the same time, control the treatment component 400 to generate radiotherapy rays, to achieve treating the patient's lesion continuously at more angles. In some embodiments, the controller may control the pedestal 100 to rotate around the third rotation axis A3, and/or control the rotation base 200 to rotate around the second rotation axis A2. When the treatment component 400 is rotated in place, the controller may control the rotation base 200 and the pedestal 100 to stop rotating, and then control the treatment component 400 to be turned on to perform positioning treatment on the patient's lesion.

In some embodiments of the present disclosure, a system is provided. The system may include one or more processors, and a memory configured to store one or more programs. When the one or more programs are executed by the one or more processors, the one or more processors are made to implement operations. The operations may include determining one or more target positions of at least one of an imaging component or a treatment component of a device. In some embodiments, the device may include a rotation base and a pedestal. The operation may further include controlling the at least one of the imaging component or the treatment component to rotate around at least one of a first rotation axis, a second rotation axis, and a third rotation axis based on the one or more target positions; and controlling the imaging component to perform imaging or the treatment component to generate radiotherapy rays. In some embodiments, the first rotation axis may be of the rotation base, the second rotation axis may be parallel with the pedestal and the rotation base, and the third rotation axis may be perpendicular to a surface of the pedestal. More descriptions for the device may be found elsewhere in the present disclosure. See, FIG. 2-FIG. 10.

In some embodiments of the present disclosure, a non-transitory computer readable medium is provided. The non-transitory computer readable medium may include a set of instructions. When the set of instructions are executed by at least one processor, the set of instructions may direct the at least one processor to perform the following acts. The acts may include determining one or more target positions of at least one of an imaging component or a treatment component of a device. In some embodiments, the device may include a rotation base and a pedestal. The acts may further include controlling the at least one of the imaging component or the treatment component to rotate around at least one of a first rotation axis, a second rotation axis, and a third rotation axis based on the one or more target positions; and controlling the imaging component to perform imaging or the treatment component to generate radiotherapy rays. In some embodiments, the first rotation axis may be of the rotation base, the second rotation axis may be parallel with the pedestal and the rotation base, and the third rotation axis may be perpendicular to a surface of the pedestal. More descriptions for the device may be found elsewhere in the present disclosure. See, FIG. 2-FIG. 10.

The possible beneficial effects of the embodiments of the present application may include but not limited to that:
(1) according to the radiation assembly in some embodiments of the present disclosure, the imaging component and the treatment component may be disposed on the same end of the rotation base. When the rotation base rotates around the first rotation axis, the imaging component may scan the patient's body to obtain an image of the patient's body and locate the lesion in the patient's body based on the image of the patient's body. The patient's lesion may be treated by the treatment component, the whole operation process of which is simple and convenient, speeding up the treatment efficiency. Moreover, the pedestal of the radiation assembly may drive the rotation base to rotate around the third rotation axis, which can enlarge the scanning angle and the treatment angle of the imaging component and the treatment component, make the image of the lesion more comprehensive, treat the lesion more thoroughly, and reduce the probability of patient recurrence;
(2) by rotating the rotation base around the first rotation axis and the pedestal around the third rotation axis, the treatment and scanning range of the imaging component and the treatment component may be expanded to a non-coplanar and multi-angle treatment and scanning range, such that the patient's lesion can receive a higher radiation dose, while the surrounding normal tissue receives a lower radiation dose, which improves the positioning accuracy of the patient's lesion, thereby protecting the normal tissue of the human body. The imaging component and the treatment component may irradiate all sides of the patient's lesion through the non-coplanar and multi-angle treatment without moving the patient, thus shortening the treatment time and improving the treatment efficiency;
(3) by controlling the rotation angle θ of the pedestal around the third rotation axis to not exceed 90°, it is possible to avoid the excessive rotation angle of the pedestal that may cause colliding with the patient's examination bed or containing cylinder;
(4) by rotating components, the rotation base may be tilted relative to the pedestal and rotate around the second rotation axis, which can increase the scanning angle and the treatment angle of the imaging component and the treatment component, thereby making the image of the lesion more comprehensive, treating the lesion more thoroughly, and reducing the risk of the probability of recurrence of the patient;
(5) the second rotation axis may intersect the first rotation axis. When the rotation base rotates around the second rotation axis and the first rotation axis, the imaging component and the treatment component may be driven to rotate around an intersection of the second rotation axis and the first rotation axis, which is conducive to scanning at various angles centered on the patient's lesion;
(6) the first rotation axis, the second rotation axis and the third rotation axis may intersect at the same point, such that the imaging component and the treatment component may achieve rotating in three directions based on the same point, and perform accurate and comprehensive scanning and treatment;
(7) by accommodating the radiation assemblies through the housing, the radiation assembly may be protected and prevented from dust.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.”

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claimed subject matter may lie in less than all features of a single foregoing disclosed embodiment.

What is claimed is:

1. A device, comprising:
a pedestal;
a rotation base disposed on the pedestal;
an imaging component disposed on the rotation base; and
a treatment component disposed on the rotation base,
wherein the imaging component and the treatment component are disposed on a same side of the rotation base, and the imaging component and the treatment component are configured to rotate around a first rotation axis of the rotation base.

2. The device of claim 1, wherein an isocenter of the imaging component and an isocenter of the treatment component is at a same position.

3. The device of claim 1, wherein the imaging component and the treatment component are located at a same rotation unit of the rotation base.

4. The device of claim 1, further comprising a first drive component configured to drive the rotation base to rotate around the first rotation axis of the rotation base.

5. The device of claim 1, further comprising a rotation component disposed between the pedestal and the rotation base, and the imaging component and the treatment component are configured to rotate, by the rotation component, around a second rotation axis parallel with the rotation base.

6. The device of claim 5, wherein an angle between the second rotation axis and a surface of the pedestal ranges from 0 to 5°.

7. The device of claim 5, wherein a rotation angle of the rotation base rotating, by the rotation component, around the second rotation axis ranges from 5° to 45°.

8. The device of claim 5, wherein a rotation angle of the rotation base rotating, by the rotation component, around the second rotation axis ranges from 5° to 60°.

9. The device of claim 5, wherein the rotation component includes a first support unit and a second support unit disposed on the pedestal, the rotation component also includes a first rocker arm and a second rocker arm disposed on the rotation base, the first rocker arm being rotatably connected with the first support unit, and the second rocker arm being rotatably connected with the second support unit.

10. The device of claim 5, further comprising
a second drive component configured to drive the rotation base to rotate around the second rotation axis.

11. The device of claim 10, wherein the second drive component includes a pneumatic pusher, an end of the pneumatic pusher being connected with the pedestal, and another end of the pneumatic pusher being connected with the rotation base.

12. The device of claim 1, wherein the pedestal is configured to rotate around a third rotation axis, wherein the third rotation axis is perpendicular to a surface of the pedestal.

13. The device of claim 12, wherein the imaging component and the treatment component are configured to rotate around the third rotation axis along with the rotation of the pedestal.

14. The device of claim 12, wherein a rotation angle of the pedestal around the third rotation axis is less than or equal to 90° or 120°.

15. The device of claim 12, further comprising a third drive component configured to drive the pedestal to rotate around the third rotation axis.

16. The device of claim 15, wherein the pedestal includes a stator and a rotor, the third drive component includes a motor and a conveyor, the motor being connected with the conveyor, and the conveyor being wounded on the rotor.

17. The device of claim 1, wherein the rotation base includes a first rotation unit and a second rotation unit, the treatment component being disposed on the first rotation unit, the imaging component being disposed on the second rotation unit, and the second rotation unit being rotatable with respect to the first rotation unit.

18. The device of claim 1, wherein the imaging component includes an imaging radiation source, and the treatment component includes a treatment radiation source, the imaging radiation source and the treatment radiation source being disposed on the rotation base at intervals.

19. A device for a medical device, comprising:
a pedestal;
a rotation base disposed on the pedestal;
an imaging component disposed on the rotation base;
a treatment component disposed on the rotation base; and
a rotation component disposed between the pedestal and the rotation base,
wherein the imaging component and the treatment component are configured to rotate, by the rotation component, around a rotation axis parallel with the pedestal and the rotation base.

20. A device for a medical device, comprising:
a pedestal;
a rotation base disposed on the pedestal;
an imaging component disposed on the rotation base; and
a treatment component disposed on the rotation base;
wherein the imaging component and the treatment component are configured to rotate around a rotation axis, the rotation axis being perpendicular to a surface of the pedestal.
